(19) United States
(12) Patent Application Publication
(10) Pub. No.: US 2024/0270970 A1
(43) Pub. Date: Aug. 15, 2024

(54) METHOD OF MANIPULATING HYDROPHILICITY AND HYDROPHOBICITY OF CONVENTIONAL DYE MOLECULES FOR TRACER APPLICATIONS

(71) Applicant: Saudi Arabian Oil Company, Dhahran (SA)

(72) Inventor: Wei Wang, Cambridge, MA (US)

(21) Appl. No.: 18/632,990

(22) Filed: Apr. 11, 2024

Related U.S. Application Data
(62) Division of application No. 17/548,858, filed on Dec. 13, 2021, now Pat. No. 11,999,855.

(51) Int. Cl. C09B 11/24; G01N 21/64; G01N 33/24

(52) U.S. Cl. CPC C09B 11/24; G01N 21/64; G01N 33/24; Y10T 436/13

(57) ABSTRACT

Functionalized fluorescent tracers, methods of making the tracers, and methods of using the tracers are provided. In some implementations, the fluorescent tracers include a functionalized fluorescent dye. The functionalized fluorescent dye includes an isothiocyanate-containing dye functionalized with a functional group that includes a primary amine. In some implementations, a method of tracing fluid flow in a subterranean formation includes mixing the functionalized fluorescent tracer into a fluid, flowing the tracer fluid into a subterranean formation, recovering a sample from a subterranean formation, and analyzing the sample for a fluorescent signal and a barcode functional group.

### CROSS-REFERENCE TO RELATED PATENT APPLICATIONS

This application is a divisional of and claims the benefit of priority to U.S. patent application Ser. No. 17/548,858, filed Dec. 13, 2021, the contents of which are incorporated by reference herein.

### TECHNICAL FIELD

This document relates to methods and compositions used in generating and using functionalized fluorescent tracers in drilling and oil well applications.

### BACKGROUND

Tracer techniques can be a powerful diagnostic tool in numerous scientific disciplines and for technologies in many industrial sectors. Molecular tracers can include water-soluble or oil-soluble compounds. In field tests of oilfields, water-soluble tracers can provide a better understanding of the studied oil reservoir, for example, a better understanding of inter-well connections, connections between layers and heterogeneities. Similarly, oil-soluble tracers can provide information on petroleum products, for example qualitative analysis of the production fluid return from multiple stage completions, either vertical or horizontal completions.

### SUMMARY

This disclosure describes functionalized fluorescent tracers, methods of making the tracers, and methods of using the tracers.

In some implementations, a composition includes a functionalized fluorescent dye. The functionalized fluorescent dye includes an isothiocyanate-containing dye functionalized with a functional group that includes a primary amine. The isothiocyanate-containing dye is selected from the group consisting of fluorescein isothiocyanate, Rhodamine B isothiocyanate, or tetramethylrhodamine isothiocyanate, or any isoform thereof. The functional group comprises a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, and hexadecylamine. Group II consists of aniline, benzylamine, phenethylamine, 3-phenyl-1-propylamine, 4-phenylbutylamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine.

In some implementations, method of making a functionalized fluorescent dye, includes dissolving a water-soluble isothiocyanate-containing fluorescent dye in an aqueous solvent to yield an aqueous dye solution. The water-soluble isothiocyanate-containing fluorescent dye is selected from the group consisting of fluorescein isothiocyanate, Rhodamine B isothiocyanate, or tetramethylrhodamine isothiocyanate, or any isoform thereof. The method includes dissolving a functional group including a primary amine in an organic solvent to yield an organic functional group solution. The functional group that includes a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, and hexadecylamine. Group II consists of aniline, benzylamine, phenethylamine, 3-phenyl-1-propylamine, 4-phenylbutylamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine. The method includes forming an emulsion of the aqueous dye solution and the organic functional group solution, and extracting the functionalized fluorescent dye from the organic functional group solution, wherein the functionalized fluorescent dye is a reaction product of the water-soluble isothiocyanate-containing dye and the functional group that includes a primary amine.

In some implementations, a method of tracing fluid flow in a subterranean formation includes mixing a functionalized fluorescent tracer into a fluid to yield a tracer fluid. The functionalized fluorescent tracer includes an isothiocyanate-containing fluorescent dye functionalized with a functional group that includes a primary amine. The isothiocyanate-containing fluorescent dye is selected from the group consisting of fluorescein isothiocyanate, Rhodamine B isothiocyanate, or tetramethylrhodamine isothiocyanate, or any isoform thereof. The functional group that includes a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, hexadecylamine, and octadecylamine. Group II consists of aniline, benzylamine, phenethylamine, 3-phenyl-1-propylamine, 4-phenylbutlamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine. The method includes flowing the tracer fluid into a first subterranean formation, recovering a sample from the first subterranean formation or a second subterranean formation, analyzing the sample for a fluorescent signal, and further separating the sample and analyzing the sample for a barcode functional group.

The details of one or more implementations of the disclosure are set forth in the accompanying drawings and the description that follows. Other features, objects, and advantages of the disclosure will be apparent from the description and drawings, and from the claims.

### DESCRIPTION OF DRAWINGS

FIG. 1 shows an example of a fracture treatment for a well.

FIG. 2 is a flow chart of an example method of making a functionalized fluorescent dye.

FIG. 3 is a flow chart of an example method of tracing fluid flow in a subterranean formation.

FIG. 4 shows the reaction product of fluorescein isothiocyante and 1-octylamine.

FIG. 5 shows the reaction product of Rhodamine B isothiocyanate and benzylamine.

FIG. 6 shows the reaction product of tetramethylrhodamine isothiocyanate and 3,4-difluoraniline.

Like reference symbols in the various drawings indicate like elements.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

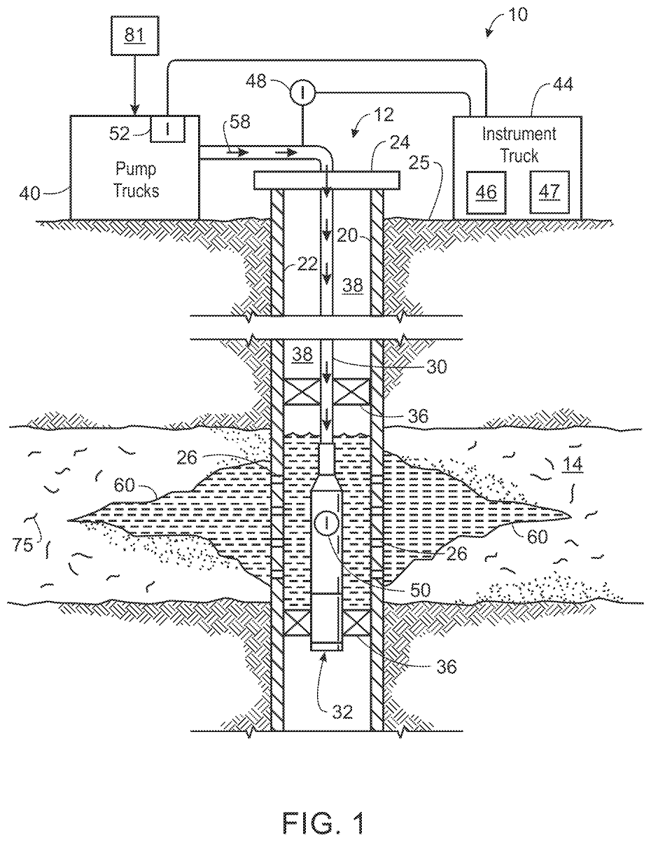

FIG. 1 illustrates an example of a drilling operation 10 for a well 12. The well 12 can be in a wellbore 20 formed in a subterranean zone 14 of a geological formation in the Earth's crust. The subterranean zone 14 can include, for example, a formation, a portion of a formation, or multiple formations in a hydrocarbon-bearing reservoir from which recovery operations can be practiced to recover trapped hydrocarbons. Examples of unconventional reservoirs include tight-gas sands, gas and oil shales, coalbed methane, heavy oil and tar sands, gas-hydrate deposits, to name a few. In some implementations, the subterranean zone 14 includes an underground formation including natural fractures 60 in rock formations containing hydrocarbons (for example, oil, gas, or both). For example, the subterranean zone 14 can include a fractured shale. In some implementations, the well 12 can intersect other suitable types of formations, including reservoirs that are not naturally fractured in any significant amount.

The well 12 can include a casing 22 and well head 24. The wellbore 20 can be a vertical, horizontal, deviated, or multilateral bore. The casing 22 can be cemented or otherwise suitably secured in the wellbore 20. Perforations 26 can be formed in the casing 22 at the level of the subterranean zone 14 to allow oil, gas, and by-products to flow into the well 12 and be produced to the surface 25. Perforations 26 can be formed using shape charges, a perforating gun, or otherwise.

For a drilling treatment 10, a work string 30 can be disposed in the wellbore 20. The work string 30 can be coiled tubing, sectioned pipe, or other suitable tubing. A drilling tool or drill bit 32 can be coupled to an end of the work string 30. Packers 36 can seal an annulus 38 of the wellbore 20 uphole of and downhole of the subterranean zone 14. Packers 36 can be mechanical, fluid inflatable, or other suitable packers.

One or more pump trucks 40 can be coupled to the work string 30 at the surface 25. The pump trucks 40 pump drilling mud 58 down the work string 30 to lubricate and cool the drilling tool or drill bit 32, maintain hydrostatic pressure in the wellbore, and carry subterranean cuttings to the surface. The drilling mud 58 can include a fluid pad, proppants, flush fluid, or a combination of these components. The pump trucks 40 can include mobile vehicles, equipment such as skids, or other suitable structures.

One or more instrument trucks 44 can also be provided at the surface 25. The instrument truck 44 can include a drilling control system 46 and a drilling simulator 47. The drilling control system 46 monitors and controls the drilling treatment 10. The drilling control system 46 can control the pump trucks 40 and fluid valves to stop and start the drilling treatment 10. The drilling control system 46 communicates with surface and subsurface instruments to monitor and control the drilling treatment 10. In some implementations, the surface and subsurface instruments may comprise surface sensors 48, down-hole sensors 50, and pump controls 52.

Additives 81 can be mixed with drilling mud 58 or other drilling fluids and flowed through the reservoir. In some implementations, the additives 81 can include one or more tracers, for example, a fluorescent dye. Hydrophilic fluorescent dyes can be used as water-soluble tracers. These conventional dyes are inexpensive and easy to use. Examples of fluorescent dyes include fluorescein, eosin, Rhodamine, and Rhodamine-B. However, duo to their poor hydrophobicity, these hydrophilic dye tracers cannot be used as partition tracers which partition between the aqueous and the oil phase. In addition, the number of tracers is limited to the number of organic dye molecules available.

The tracers described herein overcome these shortcomings. These tracers can be generated using a synthetic method to tune the hydrophilicity/hydrophobicity of water-soluble dye molecules by chemically modifying the molecular structure of the molecule. For example, by covalently grafting functional groups onto the dye molecules, the various functional groups can create barcoded structural information, resulting in new compounds. In some embodiments, by introducing hydrophobic functional groups into water-soluble dye molecules, the hydrophobicity of the resulting molecules can be enhanced, thus improving their solubility in an oil phase. By tailoring the molecules, the hydrophilicity and hydrophobicity of the molecule can be adjusted to a desired degree. Therefore, the partition of the molecule in an oil phase is controllable. This, in part, enables the potential application of these functionalized fluorescent dyes as partition tracers for oil reservoir applications. The structure-modified dyes reserve their fluorescence properties, although in some implementations the fluorescence features may also be modified by the introduction of functional groups. In some implementations, a fluorinated hydrophobic functional group can be introduced, resulting in dye compounds with low sorption on rock in fluids.

These functionalized fluorescent dyes are described herein as barcoded or having barcode information. In this context, “barcode” refers to the fact that these functionalized dyes or tracers are uniquely identifiable two or more orthogonal analyses. As a first factor, the tracers can be identified by their fluorescence signal, for example, by the wavelength of the emission spectrum or simply by the presence of a fluorescent signal. As a second factor, the tracers can be identified by their mass or hydrophobicity. Accordingly, the unique combinations of the different fluorophores and the different functionalization groups results in a library of barcoded tracers.

Barcoded tracers have several advantages. For example, different combinations of different tracers can be used simultaneously or in parallel to provide information about drilling operations or subterranean formations. For example, two or more uniquely identifiable tracers can be injected at two or more different drilling sites and can yield information about inter well connectivity. In another example, uniquely identifiable tracers can be injected at the same drilling site at different times, can yield temporal information about transit time, depth, or length of subterranean fractures or formations.

Further, the two-factor nature of the barcode tracers allows for an advantageous two-factor analysis. The first factor, the fluorescence signal, can be detected in an initial, rapid analysis. Accordingly, samples recovered from a drilling operation or subterranean formation can be quickly and qualitative analyzed for the presence of a fluorescence signal, i.e., a ‘yes/no’ analysis. In some implementations, this first analysis can be done on-site, and samples exhibiting a fluorescence signal can be allocated for further processing. Next, the samples exhibiting a fluorescence signal can be subsequently analyzed for mass or hydrophobicity, for example by high performance liquid chromatography (HPLC), mass spectrometry, liquid chromatography-mass spectrometry (LC-MS), or pyrolysis gas chromatography-mass spectrometry (Pyrolysis-GC-MS) analysis.

A chemical method to modify the structures of conventional dye molecules by introducing molecular barcode information and by tailoring the hydrophilicity and/or hydrophobicity of the conventional water-soluble dye molecules is described herein. The resulting compounds expand the number of dyes available for tracer applications as water-soluble tracers, oil tracers, or partition tracers.

The new dye molecules include the general structure R_{I}—X—R_{II}, where R_{I} is a fluorescent fluorophore. The fluorophore can be either hydrophilic or hydrophobic, and can be detectable by optical methods, for example, florescence imaging or molecular spectroscopy (absorbance or fluorescence). In the general structure, X can be a linking molecule. X can be selected from the group that includes C₁₋₁₈ alkylene, C₁₋₁₈ alkenylene, or C₁₋₁₈ alkynylene, where each of C₁₋₁₈ alkylene, C₁₋₁₈ alkenylene, C₁₋₁₈ alkynylene can be optionally replaced or interrupted by any one of oxygen (O), sulfur (S), or an amine (NH). In the general structure, R_{II} can be selected from the group that includes hydrogen, alkoxy, haloalkoxy (including Cl, Br, or I), aryl, or heteroaryl (with N, NH, O, and S). The R_{II} confers a molecular fingerprint or barcode structure into the new compounds. The new R_{I}—X—R_{II} compounds are detectable by spectroscopy methods, for example, UV-Visible spectroscopy (UV-Vis), fluorescence, Fourier-transform infrared spectroscopy (FTIR), Raman spectroscopy, mass spectroscopy, or chromatography (HPLC or LC-MS).

In some implementations, the fluorescent dye R_{II} is fluorescein isothiocyanate (FITC), Rhodamine B isothiocyanate (RBITC), or tetramethylrhodamine isothiocyanate (MRITC/TRITC), or any isomer thereof. The structures of FITC, RBITC, and MRITC/TRITC are shown in Table 1. These dyes are highly water-soluble, i.e., hydrophilic, and have fluorescence emissions in the visible spectral region. The excitation and emission wavelengths of these dyes are listed in Table 1.

TABLE 1
Water-Soluble Dyes and their Molecular Structure

| | | Fluorescence | |
|---|---|---|---|
| | Molecular structure; | λ_{excitation}/ | CAS Number/ |
| Dye compound | Molecular weight | λₑₘᵢₛₛᵢₒₙ | Isomers |
| Fluorescein isothiocyanate (FITC) | 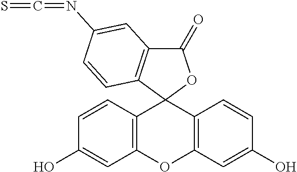 [SMILES: O=C1OC2(c3ccc(O)cc3Oc3cc(O)ccc32)c2ccc(N=C=S)cc21] MW = 389.38 | 495 nm/519nm | 27072-45-3 (mixed isomers) 3326-32-7 (5- isomer) 18861-78-4 (6-isomer) |
| Rhodamine B isothiocyanate (RBITC) | 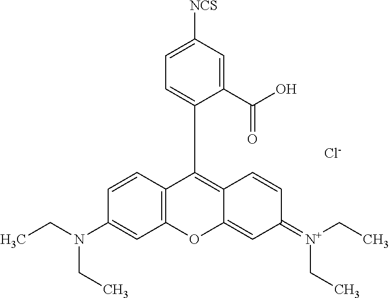 MW = 536.08 | 570 nm/ 595 nm | 36877-69-7 (mixed isomers) |
| Tetramethylrhodamine isothiocyanate (MRITC/TRITC) | 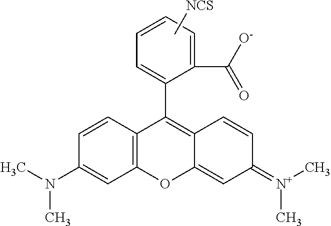 MW = 443.52 | 544 nm/ 570 nm | 95197-95-8 (mixed isomers) 80724-20-5 (Isomer R) 80724-19-2 (5-TRITC) |

The dyes shown in Table 1 were each modified with additional functional groups. The functional groups are added using the reaction of a primary amine with an isothiocyanate to result in a substituted thiourea, as shown in Equation 1.

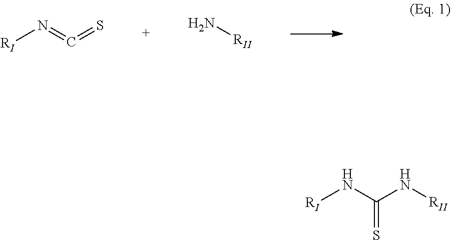

R_{II} is an alkyl, aromatic, heterocyclic group, or other suitable amine-containing functional group, and R_{I} is the isothiocyanate-containing fluorescent dye, where in Equation 1 the isothiocyanate group is expanded for clarity.

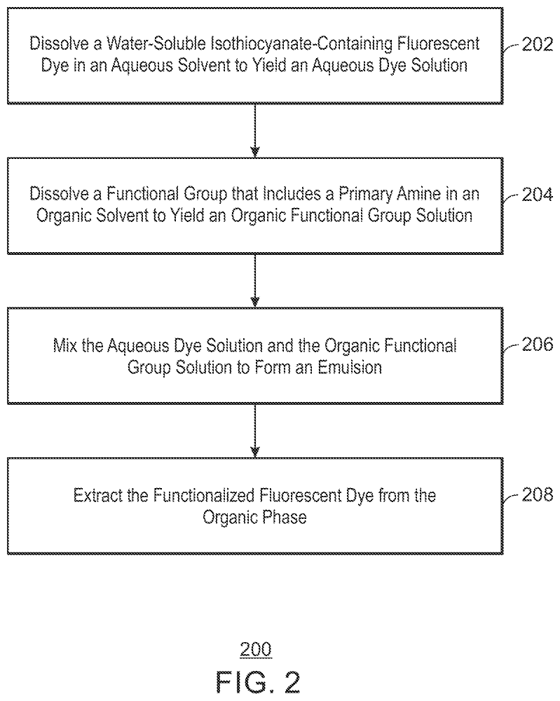

FIG. 2 is a flow chart of an example method 200 of making a functionalized fluorescent dye. The reactions were performed at room temperature in a bi-phase system. At 202, a water-soluble isothiocyanate-containing fluorescent dye is dissolved in an aqueous solvent to yield an aqueous dye solution. For example, a water-soluble isothiocyanate-containing dye can be dissolved in deionized water. At 204, a functional group that includes a primary amine is dissolved in an organic solvent to yield an organic functional group solution. For example, an amino compound can be dissolved in chloroform. The two solutions each contain the same molecular molar concentration of amino compound and dye. At 206, the aqueous dye solution and the organic functional group solution are mixed to form an emulsion. The two solutions are mixed in equal volume and stirred vigorously until the two-phase solutions formed an emulsion. The reaction is allowed to continue under stirring for at least 12 hours. Upon completion of the reaction, the stirring was stopped and the emulsion was allowed to settle into two phases. At 208, the functionalized fluorescent dye is extracted from the organic phase. It can be observed by color that the dye is transferred from the aqueous phase to the organic phase. In some implementations, the organic solvent is evaporated by N₂ bubbling or heating. The obtained solid powder samples can be redispersed into different organic solvents.

Tables 2-4 illustrate different amine-containing functional groups that can be used to functionalize an isothiocyanate dye. The amine-containing functional groups shown in Tables 2-4 can also be referred to as barcode functional groups. The compounds shown in Table 2 are primary amine compounds with alkyl groups. The hydrocarbon chains have different lengths and provide both barcode information and the ability to tailor the hydrophobicity of the resulting compound, based on, for example, alkyl chain length. Accordingly, isothiocyanate dyes functionalized with the compounds listed in Table 2 have variable partitioning in oil phases.

The compounds in Table 3 are phenyl-based compounds with hydrocarbon chains of different lengths in their structures. Compared to the compounds with alkyl chains in Table 2, the phenyl based aromatic structures with hydrocarbon chains are more hydrophobic and can have increased miscibility with oils, which provides both barcode information and the ability to tailor the hydrophobicity of the resulting compound, based on, for example, alkyl chain length. The compounds resulting from the reaction of an isothiocyanate dye and the phenyl based aromatic structures in Table 3 have variable partitioning in oil phases.

The compounds in Table 4 are fluorinated benzene based aromatic compounds. Fluorinated compounds usually have very low sorption on rocks. Compared to the compounds in Table 2 and 3, the fluorinated benzene based aromatic compounds are more hydrophobic and can have increased miscibility with oils. By introducing the fluorinated aromatic functional groups into dye molecules, the resulting functionalized dyes have variable hydrophobicity and barcode information. The functionalized dye including a fluorinated benzene based aromatic functional group have low retention in rocks and subterranean formations, and therefore can be used as partition tracers with variable hydrophobicity.

TABLE 2
Primary Amine Compounds with Alkyl Groups

| Compound | Molecular Structure/Weight (Da) | | CAS number |
|---|---|---|---|
| Ethylamine | CH₃—CH₂—NH₂ | 45.08 | 75-04-7 |
| Propylamine | CH₃—(CH₂)₂—NH₂ | 59.11 | 107-10-8 |
| Isopropylamine | (CH₃)₂—CH—NH₂ | 59.11 | 75-31-0 |
| Butylamine | CH₃—(CH₂)₃—NH₂ | 73.14 | 109-73-9 |
| tert-Butylamine | (CH₃)₃—C—NH₂ | 73.14 | 75-64-9 |
| Hexylamine | CH₃—(CH₂)₅—NH₂ | 101.19 | 111-26-2 |
| Octylamine | CH₃—(CH₂)₇—NH₂ | 129.24 | 111-86-4 |
| Decylamine | CH₃—(CH₂)₉—NH₂ | 157.30 | 2016-57-1 |
| Dodecylamine | CH₃—(CH₂)₁₁—NH₂ | 185.35 | 124-22-1 |
| Tetradecylamine | CH₃—(CH₂)₁₃—NH₂ | 213.40 | 2016-42-4 |
| Hexadecylamine | CH₃—(CH₂)₁₅—NH₂ | 241.46 | 143-27-1 |
| Octadecylamine | CH₃—(CH₂)₁₇—NH₂ | 269.51 | 124-30-1 |

TABLE 3
Phenyl Based Aromatic Compounds with Primary Amino Groups

| | | CAS |
|---|---|---|
| Compound | Molecular Structure/Weight (Da) | number |
| Aniline | 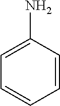 93.13 | 62-53-3 |
| Benzylamine | 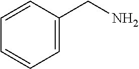 107.15 | 100-46-9 |
| Phene- thylamine | 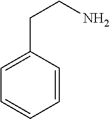 121.18 | 64-04-0 |
| 3-Phenyl-1- propylamine | 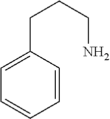 135.21 | 2038-57-5 |
| 4- Phenyl- butylamine | 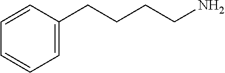 [SMILES: NCCCCc1ccccc1] 149.24 | 13214- 66-9 |
| 6- phenylhexan- 1-amine | 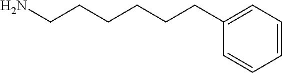 [SMILES: NCCCCCCc1ccccc1] 177.291 | 17734- 20-2 |

TABLE 4
Fluorinated Benzene Based Aromatic Compounds
with Primary Amino Group

| | | CAS |
|---|---|---|
| | Molecular Structure/ | number |
| Compound | Weight (Da) | (Isomers) |
| 4-Fluoroaniline | 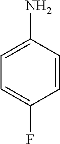 111.12 | 371-40-4 348-54-9 372-19-0 |
| 3,4-Difluoroaniline | 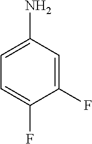 129.11 | 3863-11-4 4519-40-8 372-39-4 367-30-6 367-25-9 5509-65-9 |
| 2,4,6-Trifluoroaniline | 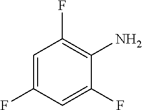 147.10 | 363-81-5 163733-96-8 3862-73-5 |
| 4- (Trifluoromethyl)aniline | 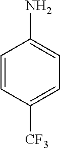 [SMILES: Nc1ccc(C(F)(F)F)cc1] 161.12 | 455-14-1 98-16-8 88-17-5 |
| 3,5- Bis(trifluoromethyl)aniline | 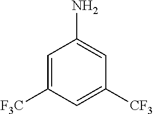 [SMILES: Nc1cc(C(F)(F)F)cc(C(F)(F)F)c1] 229.12 | 328-74-5 328-93-8 |
| 2,4,6- Tris(trifluoromethyl)aniline | 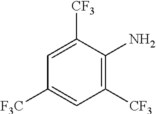 [SMILES: Nc1c(C(F)(F)F)cc(C(F)(F)F)cc1C(F)(F)F] 297.12 | 25753-22-4 |
| 4-Fluoro-3- (trifluoromethyl)aniline | 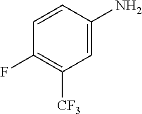 [SMILES: Nc1ccc(F)c(C(F)(F)F)c1] 179.11 | 2357-47-3 |
| 4- (Trifluoromethoxy)aniline | 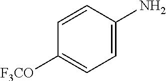 [SMILES: Nc1ccc(OC(F)(F)F)cc1] 177.12 | 461-82-5 1535-73-5 1535-75-7 |
| 4- Fluorobenzylamine | 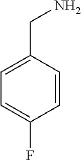 125.14 | 140-75-0 100-82-3 89-99-6 |
| 2,4- Difluorobenzylamine | 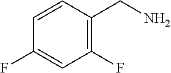 143.13 | 72235-52-0 72235-53-1 69385-30-4 72235-51-9 |
| 2,3,4- Trifluorobenzylamine | 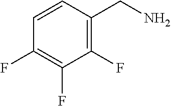 [SMILES: NCc1ccc(F)c(F)c1F] 161.12 | 235088-67-2 235088-69-4 |
| 3- (Trifluoromethyl)- benzylamine | 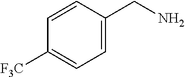 [SMILES: NCc1ccc(C(F)(F)F)cc1] 175.15 | 3300-51-4 2740-83-2 |
| 3,5- Bis(trifluoromethyl)- benzylamine | 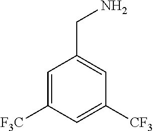 [SMILES: NCc1cc(C(F)(F)F)cc(C(F)(F)F)c1] 243.15 | 85068-29-7 |

The dyes described herein can be used as partition tracers in subterranean applications. For example, multistage hydraulic fracturing along a horizontal well is key to effectively recover hydrocarbons from tight reservoirs. Improving the hydrocarbon recovery requires detailed production information of each hydraulic fracture. Water-soluble chemical tracers are often used to calculate the production profile from multistage fracturing through a tracer flow back test, whereas oil-soluble tracers are used as a direct indicator to estimate the oil production contribution in individual fractures stages, for example, diagnosis of multi-zone oil flow efficiency, confirming zonal oil flow, or qualifying flow assurance. Oil-soluble tracers can also be embedded in the porous media and absorbed on the surfaces of solid carriers, which allows the tracers to be released from their carriers when oil passes through and has negligible partitioning into the water or gas phase. Partition tracers can well control the partition of a tracer between the aqueous and the oil phase and thus monitor the water phase and oil phase simultaneously.

With the barcoded oil-soluble partition tracers described herein, qualitative analysis by fluorescence spectroscopy or imaging can be used for early screening if the tracer is in the oil flow from each stage, while detailed molecular barcode information can be revealed by HPLC, LC-MS or Pyrolysis-GC-MS analysis to identify each tracer from different locations. Further, these synthesized barcoded oil-soluble compounds can also be added to mud formulations in drilling fluids as mud tracers for mud logging applications.

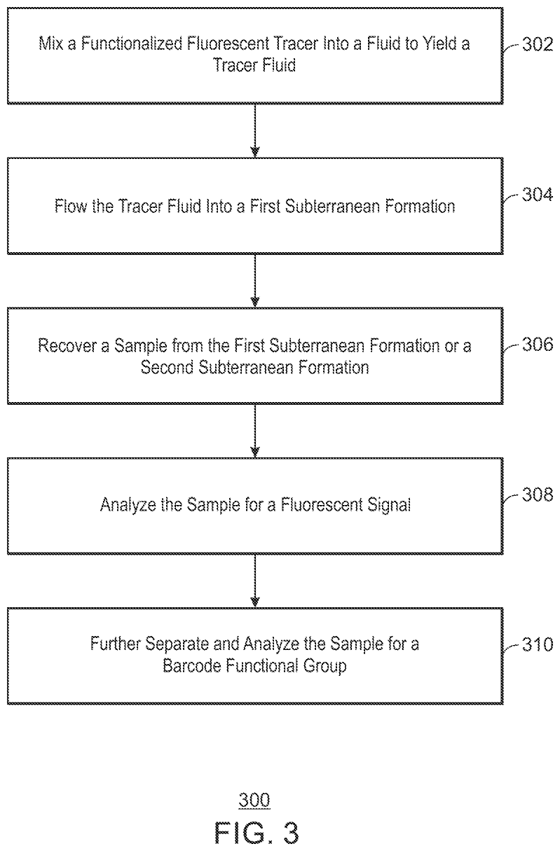

FIG. 3 is a flow chart of an example method 300 of tracing fluid flow in a subterranean formation. At 302, a functionalized fluorescent tracer is mixed into a fluid to yield a tracer fluid. At 304, the tracer fluid is flowed into a first subterranean formation. At 306, a sample is recovered from the first subterranean formation or a second subterranean formation. At 308, the sample is analyzed for a fluorescent signal. At 310, the sample is further separated and analyzed for a barcode functional group.

### EXAMPLE 1: SYNTHESIS OF FITC FUNCTIONALIZED WITH 1-OCTYLAMINE

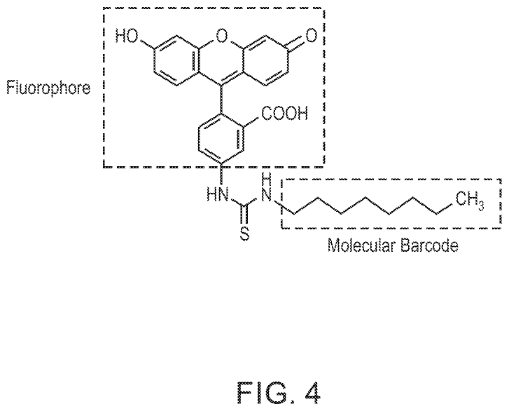

38.9 mg of FITC was dissolved in 50 mL deionized water and 12.9 mg 1-octylamine was dissolved in 50 mL benzene, respectively. Then the two solutions were mixed at 1:1 molar ration of FITC and 1-octylamine in a round bottom flask and reacted for 12 hours under vigorous stirring by a magnetic stirrer. Upon completion of the reaction, the reaction mixture was transferred into a separating funnel and left to sit overnight for phase separation. The water phase and benzene phase were collected separately. With a similar procedure, the dye FITC could be replaced by other dye molecules listed in Table 1, and the 1-octylamine can be replaced by other amines listed in Table 2. The structure of resulting isothiocyanate-amine derivative of FITC and 1-octylamine is shown in FIG. 4.

### EXAMPLE 2: SYNTHESIS OF RBITC FUNCTIONALIZED WITH BENZYLAMINE

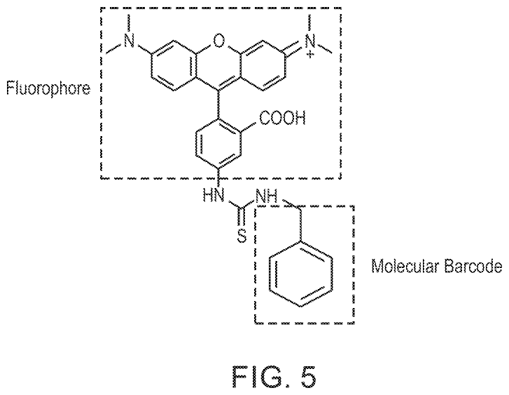

53.6 mg of RBITC was dissolved in 50 mL of a deionized water, and 10.7 mg benzylamine was dissolved in 50 mL chloroform, respectively. Then, the two solutions were mixed at 1:1 molar ration of RBITC and benzylamine in a round bottom flask and reacted for 12 hours under vigorous stirring by a magnetic stirrer. Upon completion of the reaction, the reaction mixture was transferred into a separating funnel and left to sit overnight for phase separation. The chloroform phase and water phase was separated and collected respectively. With a similar procedure, the dye FITC could be replaced by other dye molecules listed in Table 1 and the benzylamine could be replaced by other benzene-based amine compounds listed in Table 3. The structure of resulting isothiocyanate-amine derivative of BRITC and benzylamine is shown in FIG. 5.

### EXAMPLE 3: SYNTHESIS OF TRITC FUNCTIONALIZED WITH 3,4,-DIFLUOROANILINE

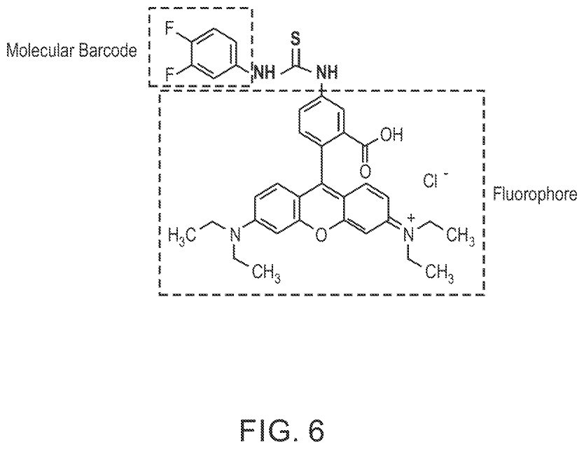

44.4 mg of TRITC was dissolved in 50 mL of deionized water, and 12.9 mg of 3,4-difluoroaniline was dissolved in 50 mL dichloromethane, respectively. Then, the two solutions were mixed at 1:1 molar ratio of TRITC and 3,4,-difluoroaniline in a round bottom flask and reacted for 12 hours under vigorous stirring by a magnetic stirrer. Upon completion of the reaction, the reaction mixture was transferred into a separating funnel and left to sit overnight for phase separation. The dichloromethane phase and water were collected separately. With a similar procedure, the dye TRITC could be replaced by the dye molecules listed in Table 1, and the 3,4-difluoroaniline could be replaced by other fluorinated aromatic compounds listed in Table 4. The structure of resulting isothiocyanate-amine derivative of TRITC and 3,4-difluoroaniline is shown in FIG. 6.

The following units of measure have been mentioned in this disclosure:

| Unit of Measure | Full form |
|---|---|
| nm | Nanometers |
| Da | Daltons |

In some implementations, a composition includes a functionalized fluorescent dye. The functionalized fluorescent dye includes an isothiocyanate-containing dye functionalized with a functional group that includes a primary amine. The isothiocyanate-containing dye is selected from the group consisting of fluorescein isothiocyanate, Rhodamine B isothiocyanate, or tetramethylrhodamine isothiocyanate, or any isoform thereof. The functional group comprises a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, and hexadecylamine. Group II consists of aniline, benzylamine, phenethylamine, 3-phenyl-1-propylamine, 4-phenylbutylamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine.

This aspect, taken alone or combinable with any other aspect, can include the following features. The isothiocyanate-containing dye is fluorescein isothiocyanate, and the functional group that includes a primary amine is selected from Group I.

This aspect, taken alone or combinable with any other aspect, can include the following features. The isothiocyanate dye is fluorescein isothiocyanate, and the functional group that includes a primary amine is octylamine.

This aspect, taken alone or combinable with any other aspect, can include the following features. The isothiocyanate-containing dye is fluorescein isothiocyanate, and the functional group that includes a primary amine is selected from Group II.

This aspect, taken alone or combinable with any other aspect, can include the following features. The isothiocyanate-containing dye is fluorescein isothiocyanate, and the functional group that includes a primary amine is selected from Group III.

This aspect, taken alone or combinable with any other aspect, can include the following features. The isothiocyanate-containing dye is Rhodamine B isothiocyanate, and the functional group including a primary amine is selected from Group I.

This aspect, taken alone or combinable with any other aspect, can include the following features. The isothiocyanate-containing dye is Rhodamine B isothiocyanate, and the functional group that includes a primary amine is selected from Group II.

This aspect, taken alone or combinable with any other aspect, can include the following features. The isothiocyanate-containing dye is Rhodamine B isothiocyanate, and the functional group that includes a primary amine is benzylamine.

This aspect, taken alone or combinable with any other aspect, can include the following features. The isothiocyanate-containing dye is Rhodamine B isothiocyanate, and the functional group that includes a primary amine is selected from Group III.

This aspect, taken alone or combinable with any other aspect, can include the following features. The isothiocyanate-containing dye is tetramethylrhodamine isothiocyanate and the functional group that includes a primary amine is selected from Group I.

This aspect, taken alone or combinable with any other aspect, can include the following features. The isothiocyanate-containing dye is tetramethylrhodamine isothiocyanate and the functional group that includes a primary amine is selected from Group II.

This aspect, taken alone or combinable with any other aspect, can include the following features. The isothiocyanate-containing dye is tetramethylrhodamine isothiocyanate and the functional group that includes a primary amine is selected from Group III.

This aspect, taken alone or combinable with any other aspect, can include the following features. The isothiocyanate-containing dye is tetramethylrhodamine isothiocyanate and the functional group that includes a primary amine is 3,4-difluoroaniline.

In some implementations, method of making a functionalized fluorescent dye, includes dissolving a water-soluble isothiocyanate-containing fluorescent dye in an aqueous solvent to yield an aqueous dye solution. The water-soluble isothiocyanate-containing fluorescent dye is selected from the group consisting of fluorescein isothiocyanate, Rhodamine B isothiocyanate, or tetramethylrhodamine isothiocyanate, or any isoform thereof. The method includes dissolving a functional group including a primary amine in an organic solvent to yield an organic functional group solution. The functional group that includes a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, and hexadecylamine. Group II consists of aniline, benzylamine, phenethylamine, 3-phenyl-1-propylamine, 4-phenylbutylamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine. The method includes forming an emulsion of the aqueous dye solution and the organic functional group solution, and extracting the functionalized fluorescent dye from the organic functional group solution, wherein the functionalized fluorescent dye is a reaction product of the water-soluble isothiocyanate-containing dye and the functional group that includes a primary amine.

This aspect, taken alone or combinable with any other aspect, can include the following features. The aqueous solvent is deionized water.

This aspect, taken alone or combinable with any other aspect, can include the following features. The organic solvent is chloroform.

This aspect, taken alone or combinable with any other aspect, can include the following features. Extracting the functionalized fluorescent dye from the organic functional group solution includes evaporating the organic solvent.

In some implementations, a method of tracing fluid flow in a subterranean formation includes mixing a functionalized fluorescent tracer into a fluid to yield a tracer fluid. The functionalized fluorescent tracer includes an isothiocyanate-containing fluorescent dye functionalized with a functional group that includes a primary amine. The isothiocyanate-containing fluorescent dye is selected from the group consisting of fluorescein isothiocyanate, Rhodamine B isothiocyanate, or tetramethylrhodamine isothiocyanate, or any isoform thereof. The functional group that includes a primary amine is selected from Group I, Group II, or Group III. Group I consists of ethylamine, propylamine, isopropylamine, butylamine, tert-butylamine, hexylamine, octylamine, decylamine, dodecylamine, tetradecylamine, hexadecylamine, and octadecylamine. Group II consists of aniline, benzylamine, phenethylamine, 3-phenyl-1-propylamine, 4-phenylbutlamine, and 6-phenylhexan-1-amine. Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine. The method includes flowing the tracer fluid into a first subterranean formation, recovering a sample from the first subterranean formation or a second subterranean formation, analyzing the sample for a fluorescent signal, and further separating the sample and analyzing the sample for a barcode functional group.

This aspect, taken alone or combinable with any other aspect, can include the following features. The method includes identifying the functionalized fluorescent tracer in the sample using fluorescence, HPLC, LC-MS, or pyrolysis-GC-MS, or any combination thereof.

This aspect, taken alone or combinable with any other aspect, can include the following features. The sample is a fluid sample or a solid sample, and wherein the fluid is a fracking fluid or a drilling mud.

The term “about” as used in this disclosure can allow for a degree of variability in a value or range, for example, within 10%, within 5%, or within 1% of a stated value or of a stated limit of a range.

The term “substantially” as used in this disclosure refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “solvent” as used in this disclosure refers to a liquid that can dissolve a solid, another liquid, or a gas to form a solution. Non-limiting examples of solvents are silicones, organic compounds, water, alcohols, ionic liquids, and supercritical fluids.

The term “room temperature” as used in this disclosure refers to a temperature of about 15 degrees Celsius (° C.) to about 28° C.

The term “downhole” as used in this disclosure refers to under the surface of the earth, such as a location within or fluidly connected to a wellbore.

As used in this disclosure, the term “drilling fluid” refers to fluids, slurries, or muds used in drilling operations downhole, such as during the formation of the wellbore.

As used in this disclosure, the term “fluid” refers to liquids and gels, unless otherwise indicated.

As used in this disclosure, the term “subterranean material” or “subterranean zone” or “subterranean formation” refers to any material under the surface of the earth, including under the surface of the bottom of the ocean. For example, a subterranean zone or material can be any section of a wellbore and any section of a subterranean petroleum- or water-producing formation or region in fluid contact with the wellbore. Placing a material in a subterranean zone can include contacting the material with any section of a wellbore or with any subterranean region in fluid contact the material. Subterranean materials can include any materials placed into the wellbore such as cement, drill shafts, liners, tubing, casing, or screens; placing a material in a subterranean zone can include contacting with such subterranean materials. In some examples, a subterranean zone or material can be any downhole region that can produce liquid or gaseous petroleum materials, water, or any downhole section in fluid contact with liquid or gaseous petroleum materials, or water. For example, a subterranean zone or material can be at least one of an area desired to be fractured, a fracture or an area surrounding a fracture, and a flow pathway or an area surrounding a flow pathway, in which a fracture or a flow pathway can be optionally fluidly connected to a subterranean petroleum- or water-producing region, directly or through one or more fractures or flow pathways.

As used in this disclosure, “treatment of a subterranean zone” can include any activity directed to extraction of water or petroleum materials from a subterranean petroleum- or water-producing formation or region, for example, including drilling, stimulation, hydraulic fracturing, clean-up, acidizing, completion, cementing, remedial treatment, abandonment, aquifer remediation, identifying oil rich regions via imaging techniques, and the like.

As used in this disclosure, a “flow pathway” downhole can include any suitable subterranean flow pathway through which two subterranean locations are in fluid connection. The flow pathway can be sufficient for petroleum or water to flow from one subterranean location to the wellbore or vice-versa. A flow pathway can include at least one of a hydraulic fracture, and a fluid connection across a screen, across gravel pack, across proppant, including across resin-bonded proppant or proppant deposited in a fracture, and across sand. A flow pathway can include a natural subterranean passageway through which fluids can flow. In some implementations, a flow pathway can be a water source and can include water. In some implementations, a flow pathway can be a petroleum source and can include petroleum. In some implementations, a flow pathway can be sufficient to divert water, a downhole fluid, or a produced hydrocarbon from a wellbore, fracture, or flow pathway connected to the pathway.

As used in this disclosure, “weight percent” (wt %) can be considered a mass fraction or a mass ratio of a substance to the total mixture or composition. Weight percent can be a weight-to-weight ratio or mass-to-mass ratio, unless indicated otherwise.

A number of implementations of the disclosure have been described. Nevertheless, it will be understood that various modifications may be made without departing from the spirit and scope of the disclosure.

What is claimed is:

1. A composition comprising:
a functionalized fluorescent dye, wherein the functionalized fluorescent dye comprises an isothiocyanate-containing dye functionalized with a functional group comprising a primary amine, wherein
the isothiocyanate-containing dye is selected from the group consisting of fluorescein isothiocyanate, Rhodamine B isothiocyanate, or tetramethylrhodamine isothiocyanate, or any isoform thereof, and
the functional group comprises a primary amine is selected from Group I or Group III, wherein
Group I consists of ethylamine, propylamine, isopropylamine, butylamine, tert-butylamine, hexylamine, octylamine, decylamine, dodecylamine, tetradecylamine, and hexadecylamine, and
Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine.

2. The composition of claim 1, wherein the isothiocyanate-containing dye is fluorescein isothiocyanate, and the functional group comprising a primary amine is selected from Group I.

3. The composition of claim 2, wherein the isothiocyanate dye is fluorescein isothiocyanate, and the functional group comprising a primary amine is octylamine.

4. The composition of claim 1, wherein the isothiocyanate-containing dye is fluorescein isothiocyanate, and the functional group comprising a primary amine is selected from Group III.

5. The composition of claim 1, wherein the isothiocyanate-containing dye is Rhodamine B isothiocyanate, and the functional group comprising a primary amine is selected from Group I.

6. The composition of claim 1, wherein the isothiocyanate-containing dye is Rhodamine B isothiocyanate, and the functional group comprising a primary amine is selected from Group III.

7. The composition of claim 1, wherein the isothiocyanate-containing dye is tetramethylrhodamine isothiocyanate and the functional group comprising a primary amine is selected from Group I.

8. The composition of claim 1, wherein the isothiocyanate-containing dye is tetramethylrhodamine isothiocyanate and the functional group comprising a primary amine is selected from Group III.

9. The composition of claim 8, wherein the isothiocyanate-containing dye is tetramethylrhodamine isothiocyanate and the functional group comprising a primary amine is 3,4-difluoroaniline.

10. A method of making a functionalized fluorescent dye, comprising:
dissolving a water-soluble isothiocyanate-containing fluorescent dye in an aqueous solvent to yield an aqueous dye solution, wherein the water-soluble isothiocyanate-containing fluorescent dye is selected from the group consisting of fluorescein isothiocyanate, Rhodamine B isothiocyanate, or tetramethylrhodamine isothiocyanate, or any isoform thereof;
dissolving a functional group comprising a primary amine in an organic solvent to yield an organic functional group solution, wherein the functional group comprising a primary amine is selected from Group I, Group II, or Group III, wherein
Group I consists of ethylamine, propylamine, isopropylamine, butylamine, tert-butylamine, hexylamine, octylamine, decylamine, dodecylamine, tetradecylamine, and hexadecylamine,
Group II consists of aniline, benzylamine, phenethylamine, 3-phenyl-1-propylamine, 4-phenylbutylamine, and 6-phenylhexan-1-amine, and
Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine;
forming an emulsion of the aqueous dye solution and the organic functional group solution; and
extracting the functionalized fluorescent dye from the organic functional group solution, wherein the functionalized fluorescent dye is a reaction product of the water-soluble isothiocyanate-containing dye and the functional group comprising a primary amine.

11. The method of claim 10, wherein the aqueous solvent is deionized water.

12. The method of claim 10, wherein the organic solvent is chloroform.

13. The method of claim 10, wherein extracting the functionalized fluorescent dye from the organic functional group solution comprises evaporating the organic solvent.

14. A method of tracing fluid flow in a subterranean formation, comprising:
mixing a functionalized fluorescent tracer into a fluid to yield a tracer fluid, wherein the functionalized fluorescent tracer comprises an isothiocyanate-containing fluorescent dye functionalized with a functional group comprising a primary amine, wherein
the isothiocyanate-containing fluorescent dye is selected from the group consisting of fluorescein isothiocyanate, Rhodamine B isothiocyanate, or tetramethylrhodamine isothiocyanate, or any isoform thereof,
the functional group comprising a primary amine is selected from Group I, Group II, or Group III, wherein
Group I consists of ethylamine, propylamine, isopropylamine, butylamine, tert-butylamine, hexylamine, octylamine, decylamine, dodecylamine, tetradecylamine, hexadecylamine, and octadecylamine,
Group II consists of aniline, benzylamine, phenethylamine, 3-phenyl-1-propylamine, 4-phenylbutlamine, and 6-phenylhexan-1-amine, and
Group III consists of 4-fluoroaniline, 3,4-difluoroaniline, 2,4,6-trifluoroaniline, 4-(trifluoromethyl)aniline, 3,5-bis(trifluoromethyl)aniline, 2,4,6-tris(trifluoromethyl)aniline, 4-fluoro-3-(trifluoromethyl)aniline, 4-(trifluoromethoxy)aniline, 4-fluorobenzylamine, 2,4-difluorobenzylamine, 2,3,4-trifluorobenzylamine, 3-(trifluoromethyl)benzylamine, and 3,5-bis(trifluoromethyl)benzylamine;
flowing the tracer fluid into a first subterranean formation;
recovering a sample from the first subterranean formation or a second subterranean formation;
analyzing the sample for a fluorescent signal; and
further separating the sample and analyzing the sample for a barcode functional group.

15. The method of claim 14, further comprising identifying the functionalized fluorescent tracer in the sample using fluorescence, HPLC, LC-MS, or pyrolysis-GC-MS, or any combination thereof.

16. The method of claim 14, wherein the sample is a fluid sample or a solid sample, and wherein the fluid is a fracking fluid or a drilling mud.
